(19) United States
(12) Patent Application Publication
(10) Pub. No.: US 2024/0104430 A1
(43) Pub. Date: Mar. 28, 2024

(54) INFORMATION PROCESSING APPARATUS, FEATURE QUANTITY SELECTION METHOD, TRAINING DATA GENERATION METHOD, ESTIMATION MODEL GENERATION METHOD, STRESS LEVEL ESTIMATION METHOD, AND STORAGE MEDIUM

(71) Applicant: NEC Corporation, Minato-ku, Tokyo (JP)

(72) Inventors: Yoshiki NAKASHIMA, Tokyo (JP); Masanori TSUJIKAWA, Tokyo (JP)

(73) Assignee: NEC Corporation, Minato-ku, Tokyo (JP)

(21) Appl. No.: 18/273,456

(22) PCT Filed: Jan. 21, 2021
(86) PCT No.: PCT/JP2021/001945

§ 371 Date: Jul. 20, 2023

(51) Int. Cl. G06N 20/00; G16H 20/70

(52) U.S. Cl. CPC G06N 20/00; G16H 20/70

(57) ABSTRACT

In order to improve a feature quantity selection method for machine learning of a stress level estimation model, an information processing apparatus (1) includes: a first selection section (11) that generates a feature set by selecting a feature quantity corresponding to each of a plurality of modalities from among a plurality of feature quantities; and a second selection section (12) that selects, based on a result of verifying estimation accuracy, a combination of feature quantities for use in the machine learning, the verification being carried out by applying combinations of feature quantities included in the feature set to the machine learning of the estimation model.

### TECHNICAL FIELD

The present invention relates to feature quantity selection for machine learning of a stress level estimation model.

### BACKGROUND ART

In recent years, there are increasing cases where employees suffer from mental problems such as depression due to occupational stress, resulting in quitting jobs or taking leaves of absence. Along with the circumstances, there is also a problem of increasing burdens on companies that maintain and secure employees. Against this background, studies on stress monitoring are underway. For example, studies are also underway on a technique for generating a stress level estimation model using measurement data such as body motion data and biological data of a subject, and estimating a stress level of the subject using the generated estimation model.

Here, in regard to stress estimation, although it is assumed that many statistics calculated based on biological signals and the like are feature quantities that are effective for stress estimation, there is no clear knowledge about which is optimal. In order to construct an estimation model, it is necessary to collect, in addition to data of a feature quantity, a stress score indicating a stress level of a subject at a time when the feature quantity has been measured. Here, a cost for collecting these pieces of data is high. Therefore, data samples obtained are often less than the number of candidate feature quantities. In such a case, it is difficult to increase learning accuracy due to “curse of dimensionality”.

Although not a document disclosing a technique for stress estimation, for example, Patent Literature 1 below can be cited as a document disclosing feature quantity selection. Patent Literature 1 discloses an apparatus for determining a psychological state of a user using an inference model. In this apparatus, feature data for analyzing a psychological state of a user is extracted from sensor data measured by various sensors, and a part having a high degree of importance is selected from the extracted feature data using various feature quantity selection algorithms. Specifically, in the technique of Patent Literature 1, the degree of importance of feature data is calculated using feature quantity selection algorithms such as an information gain, a chi-square distribution, and a mutual information algorithm, and a part of feature data having a high degree of importance is selected.

### CITATION LIST

### Patent Literature

[Patent Literature 1]
Japanese Patent Application Publication Tokukai No. 2018-187441

### SUMMARY OF INVENTION

### Technical Problem

However, the above described feature quantity selection method is a general method that does not take into consideration properties of various feature quantities. Therefore, in a case where the above described feature quantity selection method is applied to machine learning of a stress level estimation model, there is room for improvement. An example aspect of the present invention is accomplished in view of this point, and its example object is to provide an information processing apparatus and the like that make it possible to improve a feature quantity selection method for machine learning of a stress level estimation model.

### Solution to Problem

An information processing apparatus according to an example aspect of the present invention includes: a first selection means that generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a second selection means that selects, based on a result of verifying estimation accuracy, a combination of feature quantities for use in the machine learning, the verification being carried out by applying combinations of feature quantities included in the feature set to the machine learning of the estimation model.

A feature quantity selection method according to an example aspect of the present invention includes: generating, by at least one processor, a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and selecting, by the at least one processor based on a result of verifying estimation accuracy, a combination of feature quantities for use in the machine learning, the verification being carried out by applying combinations of feature quantities included in the feature set to the machine learning of the estimation model.

A program according to an example aspect of the present invention causes a computer to function as: a first selection means that generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a second selection means that selects, based on a result of verifying estimation accuracy, a combination of feature quantities for use in the machine learning, the verification being carried out by applying combinations of feature quantities included in the feature set to the machine learning of the estimation model.

### Advantageous Effects of Invention

According to an example aspect of the present invention, it is possible to improve a feature quantity selection method for machine learning of a stress level estimation model.

### BRIEF DESCRIPTION OF DRAWINGS

FIG. 1 is a block diagram illustrating a configuration of an information processing apparatus according to a first example embodiment of the present invention.

FIG. 2 is a flowchart illustrating a flow of a feature quantity selection method according to the first example embodiment of the present invention.

FIG. 3 is a diagram illustrating an overview of a process carried out by an information processing apparatus according to a second example embodiment of the present invention.

FIG. 4 is a block diagram illustrating a configuration of the information processing apparatus.

FIG. 5 is a flowchart illustrating a flow of an estimation model generation method according to the second example embodiment of the present invention.

FIG. 6 is a flowchart illustrating a flow of a stress level estimation method according to the second example embodiment of the present invention.

FIG. 7 is a diagram illustrating an overview of a process carried out by an information processing apparatus according to a third example embodiment of the present invention.

FIG. 8 is a diagram illustrating a result of an experiment for verifying an effect of a feature quantity selection method according to each of example embodiments of the present invention.

FIG. 9 is a diagram illustrating an example of a computer which executes instructions of a program that is software realizing functions of the information processing apparatus according to each of example embodiments of the present invention.

### EXAMPLE EMBODIMENTS

### First Example Embodiment

The following description will discuss a first example embodiment of the present invention in detail with reference to the drawings. The present example embodiment is a basic form of example embodiments described later.

(Configuration of Information Processing Apparatus)

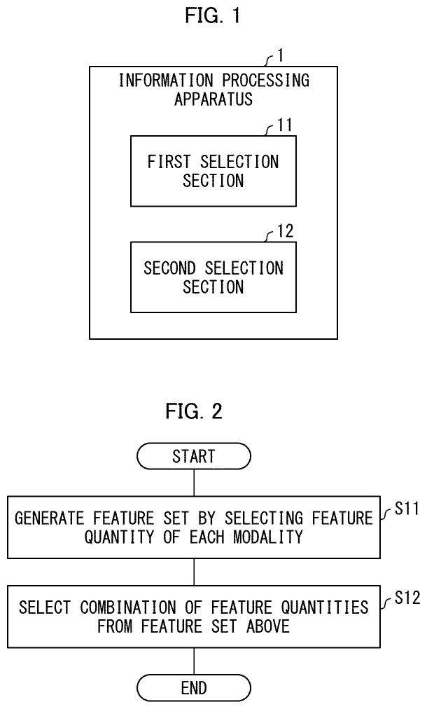

The following description will discuss a configuration of an information processing apparatus 1 according to the present example embodiment, with reference to FIG. 1. FIG. 1 is a block diagram illustrating the configuration of the information processing apparatus 1. As illustrated in FIG. 1, the information processing apparatus 1 includes a first selection section 11 and a second selection section 12.

The first selection section 11 generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level. Note that the utility of a feature quantity is utility at a time when the feature quantity is applied to machine learning. By carrying out machine learning using feature quantities having high utility, it is possible to generate an estimation model capable of carrying out estimation with high accuracy. An evaluation method of utility is not particularly limited, as long as the method can classify feature quantities into (i) a feature quantity which is highly likely to contribute to generation of an estimation model capable of carrying out highly accurate estimation and (ii) a feature quantity which is less likely to contribute to generation of such an estimation model.

The second selection section 12 selects, based on a result of verifying estimation accuracy, a combination of feature quantities for use in the machine learning, the verification being carried out by applying combinations of feature quantities included in the feature set to the machine learning of the estimation model. A verification method of estimation accuracy is not particularly limited, and any method can be employed.

As described above, the information processing apparatus 1 according to the present example embodiment employs the configuration in which a feature set is generated by selecting at least one feature quantity corresponding to each of a plurality of modalities from among a plurality of feature quantities based on respective utility evaluation results for the plurality of feature quantities. Then, a combination of feature quantities for use in machine learning of an estimation model is selected, based on a result of verifying estimation accuracy by applying combinations of feature quantities included in the generated feature set to the machine learning of the estimation model.

According to the configuration, a feature quantity is selected based on a utility evaluation result for each of a plurality of feature quantities, before verifying estimation accuracy by applying combinations of feature quantities to machine learning of an estimation model. Thus, verification of estimation accuracy is carried out with respect to a highly useful one of the plurality of feature quantities. Therefore, it is possible to carry out efficient verification and to reduce a possibility of causing a problem of “curse of dimensionality” due to an excessive number of dimensions of feature quantities used for machine learning.

Note, however, that, in feature quantity selection based on a utility evaluation result, there is a possibility that a feature quantity corresponding to any of modalities is not selected. Note that a modality of a feature quantity is a classification which is determined according to a property of the feature quantity. What kind of feature quantity is classified into what kind of modality may be determined in advance. For example, in “Physiological signal based work stress detection using unobtrusive sensors” (Anusha, et al., Biomed. Phys. Eng. Express, vol. 4, no. 6, p. 065001, September 2018), perspiration and skin temperature are classified into separate modalities in stress estimation. Moreover, “Towards an automatic early stress recognition system for office environments based on multimodal measurements” (Alberdi, et al., Journal of Biomedical Informatics, vol. 59, pp. 49-75, February 2016) indicates that signs of stress are exhibited in a multimodal manner. Specifically, it is described that signs of stress appear in three modalities: psychological, physiological, and behavioral.

According to the foregoing configuration, a feature set is generated by selecting at least one feature quantity corresponding to each of a plurality of modalities. Thus, it is possible to increase a possibility that feature quantities corresponding to respective of all the modalities are selected, and it is possible to construct an estimation model having high robustness by machine learning using the selected feature quantities. Note that the estimation model having high robustness is an estimation model that is capable of carrying out stable estimation with high accuracy.

As described above, the information processing apparatus 1 according to the present example embodiment brings about an example advantage of improving a feature quantity selection method for machine learning of a stress level estimation model.

(Flow of Feature Quantity Selection Method)

The following description will discuss a flow of a feature quantity selection method according to the present example embodiment, with reference to FIG. 2. FIG. 2 is a flowchart illustrating the flow of the feature quantity selection method.

In S11, at least one processor generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level.

In S12, the at least one processor selects, based on a result of verifying estimation accuracy, a combination of feature quantities for use in the machine learning of the estimation model, the verification being carried out by applying combinations of feature quantities included in the feature set to the machine learning of the estimation model.

Note that the processes of S11 and S12 may be carried out by a single processor, or the processes of S11 and S12 may be carried out by separate processors. In the latter case, the processors can be provided in a single information processing apparatus (e.g., the information processing apparatus 1 illustrated in FIG. 1) or can be provided in respective different information processing apparatuses.

As described above, the feature quantity selection method according to the present example embodiment employs the configuration in which the at least one processor selects, based on a result of verifying estimation accuracy, a combination of feature quantities for use in the machine learning of the estimation model, the verification being carried out by applying combinations of feature quantities included in the feature set to the machine learning of the estimation model, and the at least one processor selects, based on a result of verifying estimation accuracy, a combination of feature quantities for use in the machine learning of the estimation model, the verification being carried out by applying combinations of feature quantities included in the feature set to the machine learning of the estimation model. Therefore, the feature quantity selection method according to the present example embodiment brings about an example advantage of improving a feature quantity selection method for machine learning of a stress level estimation model.

The functions of the information processing apparatus 1 described above can also be realized by a program. A feature quantity selection program according to the present example embodiment is a program for causing a computer to function as the information processing apparatus 1, and employs the configuration in which the program causes the computer to function as: a first selection means that generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a second selection means that selects, based on a result of verifying estimation accuracy, a combination of feature quantities for use in the machine learning, the verification being carried out by applying combinations of feature quantities included in the feature set to the machine learning of the estimation model. Therefore, the feature quantity selection program according to the present example embodiment brings about an example advantage of improving a feature quantity selection method for machine learning of a stress level estimation model.

### Second Example Embodiment

(Overview)

In the present example embodiment, an example will be described in which processes from selection of feature quantities for constructing a stress level estimation model, generation of an estimation model using the selected feature quantities, and estimation of a stress level using the generated estimation model are carried out by a single information processing apparatus. The information processing apparatus is referred to as an information processing apparatus 4.

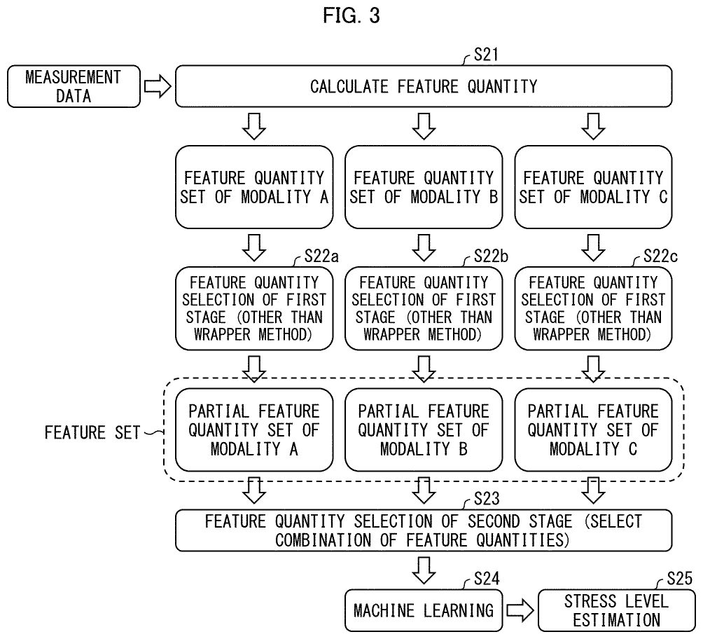

FIG. 3 is a diagram illustrating an overview of a process carried out by the information processing apparatus 4. In S21, the information processing apparatus 4 calculates a feature quantity from measurement data pertaining to a stress level that indicates a degree of stress of a subject.

In the present example embodiment, a multimodal signal is sensed by a wearable device worn by the subject. Specifically, the present example embodiment describes an example of measuring, by the wearable device, body motion data (e.g., acceleration data) indicating body motion of the subject, heart rate data indicating a heart rate of the subject, and perspiration data indicating perspiration of the subject as the measurement data. Of course, measurement data is not limited to the above three types, and only needs to be correlated with a stress level of a subject. For example, biological signal data indicating body temperature, brain waves, pulse, or the like of a subject may be used as the measurement data.

A method for calculating a feature quantity in S21 can be any method, as long as the method is capable of calculating a feature quantity related to a stress level. For example: the measurement data itself can be used as a feature quantity; a feature quantity can be obtained by removing a noise component from the measurement data; a feature quantity can be obtained by time-dividing the measurement data; or a feature quantity can be calculated by substituting the measurement data into a predetermined mathematical formula. In S21, the information processing apparatus 4 may calculate a plurality of types of feature quantities from one type of measurement data. Thus, for example, even in a case where measurement data is three types, i.e., body motion data, heart rate data, and perspiration data, it is possible to generate several hundreds to several thousands of feature quantities.

In S22a through S22c, the information processing apparatus 4 carries out feature quantity selection of a first stage. In the feature quantity selection of the first stage, feature quantity selection is carried out by a method other than a wrapper method by classifying, according to modalities, the feature quantities which have been calculated in S21. Hereinafter, a set of feature quantities of each modality is referred to as a feature quantity set of the modality.

Note that the wrapper method is one of techniques of feature quantity selection. In the wrapper method, an optimal combination of feature quantities for use in machine learning is selected based on a result of verifying estimation accuracy by applying combinations of feature quantities to machine learning of an estimation model. Meanwhile, in feature quantity selection in S22a through S22c by a method other than the wrapper method, the information processing apparatus 4 evaluates utility for each of a plurality of feature quantities and selects a feature quantity having high utility. That is, in S22a through S22c, the information processing apparatus 4 carries out, for each modality, evaluation of utility and selection of a feature quantity based on a result of the evaluation. The method other than the wrapper method is different from the wrapper method in that an estimation model is not used for evaluation. Specific examples of the method other than the wrapper method include a filter method, a principal component analysis, and the like.

In the example of FIG. 3, feature quantity selection is carried out for each of three types of modalities A through C. For example, a modality may be set for each piece of measurement data from which a feature quantity has been calculated. In this case, for example, various feature quantities calculated from body motion data may be classified into a modality A, various feature quantities calculated from heart rate data may be classified into a modality B, and various feature quantities calculated from perspiration data may be classified into a modality C. For example, feature quantities generated from physiological signals that pertain to physiological phenomena reflecting a stress state of a subject such as pulse waves, perspiration, and body temperature may be classified into a physiological modality. Moreover, feature quantities generated from behavior signals that pertain to behaviors reflecting a stress state of a subject such as body motion may be classified into a behavioral modality.

In the stage of carrying out the processes of S22a through S22c, feature quantities are not sufficiently narrowed down. Therefore, if feature quantity selection is carried out by the wrapper method at this stage, there is a concern that a processing time becomes longer and proper feature quantity selection cannot be carried out due to the curse of dimensionality. Therefore, in S22a through S22c, feature quantity selection is carried out by a method other than the wrapper method. Thus, feature quantity selection can be carried out with a smaller computation amount than that of the wrapper method, and a problem of the curse of dimensionality can also be avoided.

In S22a, the information processing apparatus 4 carries out feature quantity selection from a feature quantity set that is constituted by feature quantities of the modality A among feature quantities calculated in S21. Thus, a partial feature quantity set of the modality A is obtained, which is constituted by feature quantities of the modality A and from which feature quantities that are not useful have been screened off by the process of S22a.

Similarly, in S22b, the information processing apparatus 4 carries out feature quantity selection from a feature quantity set that is constituted by feature quantities of the modality B among feature quantities calculated in S21. Thus, a partial feature quantity set of the modality B is obtained, which is constituted by feature quantities of the modality B and from which feature quantities that are not useful have been screened off by the process of S22b.

Similarly, in S22c, the information processing apparatus 4 carries out feature quantity selection from a feature quantity set that is constituted by feature quantities of the modality C among feature quantities calculated in S21. Thus, a partial feature quantity set of the modality C is obtained, which is constituted by feature quantities of the modality C and from which feature quantities that are not useful have been screened off by the process of S22c. Note that methods of feature quantity selection in S22a through S22c may be the same or may be different from each other. The numbers of feature quantities selected in S22a through S22c may be the same or may be different from each other. Note, however, that if the number of feature quantities selected is excessively large, there arises a problem of an increase in a processing time of S23 and/or problem of the curse of dimensionality. Therefore, it is preferable that the total number of feature quantities selected in S22a through S22c falls within a range in which such problems are unlikely to occur.

Through the above described processes, it is possible to obtain a feature set including at least one feature quantity corresponding to each of the modalities A through C. In S23, a second stage of feature quantity selection is carried out from the feature set. In the second stage of feature quantity selection, the information processing apparatus 4 verifies estimation accuracy by applying combinations of feature quantities included in the feature set to machine learning of an estimation model. Then, the information processing apparatus 4 selects, based on a result of the verification, a combination of feature quantities for use in machine learning. The feature quantity selection in S23 can be carried out by use of, for example, a wrapper method. The wrapper method is a feature quantity selection technique in which a combination of feature quantities is evaluated by actually using an estimation model. Therefore, it is extremely effective for selection of a suitable combination of feature quantities.

Note, however, that the wrapper method is model-based learning. Therefore, if training is carried out by a large number of feature quantities, a learning effect may be reduced due to the curse of dimensionality, and a processing time may increase. Therefore, as described above, the information processing apparatus 4 carries out narrowing down of feature quantities by the processes of S22a through S22c. Thus, it is possible to select a suitable combination of feature quantities, and it is also possible to avoid an increase in processing time, while avoiding the curse of dimensionality.

In S24, the information processing apparatus 4 carries out machine learning using the combination of feature quantities which has been selected in S23, and generates a stress level estimation model. More specifically, in S24, the information processing apparatus 4 first generates training data for use in machine learning by associating, as correct answer data, a stress level of a subject with the combination of feature quantities which has been selected in S23. Then, the information processing apparatus 4 carries out machine learning using the generated training data and generates a stress level estimation model.

In S25, the information processing apparatus 4 estimates the stress level of the subject using the estimation model which has been generated by the machine learning in S24. More specifically, in S25, the information processing apparatus 4 calculates, from measurement data in a predetermined time period of the subject, feature quantities corresponding to the combination selected in S23 described above, and inputs the calculated feature quantities into the estimation model which has been generated by the machine learning in S24. Then, the information processing apparatus 4 estimates a stress level in the predetermined time period of the subject based on an output value of the estimation model.

As described above, the information processing apparatus 4 generates a feature set by selecting at least one feature quantity corresponding to each of a plurality of modalities from among a plurality of feature quantities which have been calculated from measurement data (S22a through S22c). Then, the information processing apparatus 4 selects, based on a result of verifying estimation accuracy, a combination of feature quantities for use in machine learning, the verification being carried out by applying combinations of feature quantities included in the generated feature set to the machine learning of the estimation model (S23).

Thus, in the first stage of feature quantity selection (S22a through S22c), a case will not occur in which feature quantities of any of modalities are missing. Then, in the second stage of feature quantity selection (S23), a suitable combination of feature quantities is selected. Therefore, in S24, it is highly likely that an estimation model is generated for which feature quantities of the modalities are used as explanatory variables. Thus, in S25, it is possible to carry out estimation with high robustness.

Note that the process of S23 may also be carried out for each modality. Thus, it is possible to reliably leave feature quantities of each modality. Moreover, the processes carried out by the information processing apparatus 4 may be carried out by sharing among a plurality of information processing apparatuses. For example, it is possible that the information processing apparatus 4 selects feature quantities, another information processing apparatus generates training data using the selected feature quantities, and still another information processing apparatus generates an estimation model using the generated training data. Then, yet another information processing apparatus may estimate a stress level of a subject by using the generated estimation model. Alternatively, it is possible that, for example, the information processing apparatus 4 carries out the processes from selection of feature quantities to generation of an estimation model, and another information processing apparatus estimates a stress level of a subject using the generated estimation model.

In a case where estimation accuracy of the estimation model generated in S24 does not satisfy a predetermined criterion, the information processing apparatus 4 may reselect feature quantities. In this case, in the processes of S22a through S22c at and after the second time, the information processing apparatus 4 selects feature quantities by an evaluation method different from that of the previous time, and generates a feature set different from that of the previous time. After that, feature quantities are selected from the feature set as described above, machine learning is carried out using the selected feature quantities, and an estimation model is generated (S23 and S24). By repeating such processes, it is possible to generate an estimation model that satisfies a predetermined criterion. Note that a method such as cross validation can be applied to evaluation of estimation accuracy of the generated estimation model.

(Configuration of Information Processing Apparatus 4)

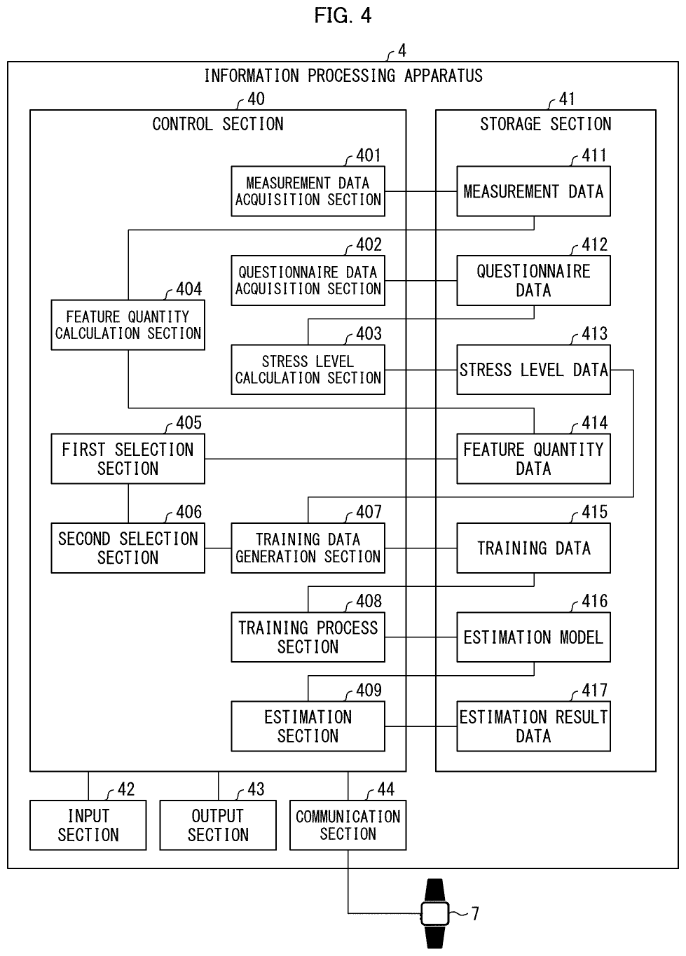

The following description will discuss a configuration of the information processing apparatus 4 with reference to FIG. 4. FIG. 4 is a block diagram illustrating the configuration of the information processing apparatus 4. FIG. 4 also illustrates a wearable terminal 7 as an example of an apparatus that measures measurement data.

The wearable terminal 7 includes a triaxial acceleration sensor and transmits an output value of the acceleration sensor as measurement data to the information processing apparatus 4. When the subject wears the wearable terminal 7, body motion of the subject is detected by the acceleration sensor. Since it has been found that body motion is correlated with the stress level of the subject, it is possible to estimate a stress level by using an output value of the acceleration sensor as measurement data. Note that the acceleration sensor is not limited to a triaxial acceleration sensor, and may be a uniaxial or biaxial acceleration sensor.

The wearable terminal 7 also has a function of detecting a heart rate of the wearer and a function of detecting perspiration of the wearer. Therefore, when the subject wears the wearable terminal 7, heart rate data and perspiration data are generated in addition to the acceleration data, and the pieces of data are transmitted to the information processing apparatus 4 as measurement data pertaining to the stress level of the subject. Here, for simplicity, an example will be described in which the wearable terminal 7 transmits all of necessary pieces of measurement data to the information processing apparatus 4. However, the information processing apparatus 4 may obtain pieces of measurement data from separate apparatuses.

The information processing apparatus 4 includes a control section 40 that comprehensively controls components of the information processing apparatus 4, and a storage section 41 that stores various kinds of data used by the information processing apparatus 4. The information processing apparatus 4 further includes: an input section 42 that receives input of data with respect to the information processing apparatus 4; an output section 43 for outputting data from the information processing apparatus 4; and a communication section 44 for carrying out communication between the information processing apparatus 4 and another apparatus (e.g., the wearable terminal 7).

The control section 40 includes a measurement data acquisition section 401, a questionnaire data acquisition section 402, a stress level calculation section 403, a feature quantity calculation section 404, a first selection section 405, a second selection section 406, a training data generation section 407, a training process section 408, and an estimation section 409. The storage section 41 stores measurement data 411, questionnaire data 412, stress level data 413, feature quantity data 414, training data 415, an estimation model 416, and estimation result data 417.

The measurement data acquisition section 401 acquires measurement data pertaining to the stress level of the subject and causes the storage section 41 to store the acquired measurement data. The measurement data stored in the storage section 41 is measurement data 411. The measurement data 411 can include data used for generation of training data 415 and data used for estimation of a stress level.

The questionnaire data acquisition section 402 acquires a result of a questionnaire pertaining to the stress level of the subject in a time period in which measurement data 411 (that is used for generation of training data 415) has been measured, and causes the storage section 41 to store questionnaire data 412 indicating the acquired result. This questionnaire is a questionnaire answered by the subject in order to calculate the stress level of the subject. The questionnaire only needs to have content that reflects a stress level of a subject, and may be a stress questionnaire of, for example, perceived stress scale (PSS). The stress questionnaire of PSS is a questionnaire in the form in which a subject selects an applicable one from a plurality of options, for each of a plurality of questions regarding how the subject feels and behaves during a time period in question.

The stress level calculation section 403 calculates a stress level of a subject using the questionnaire data 412, and causes the storage section 41 to store stress level data 413 that indicates the calculated stress level. Any method for calculating the stress level can be applied. For example, in a case where the questionnaire data 412 is data indicating a result of a stress questionnaire of PSS, the stress level calculation section 403 calculates a PSS score.

The feature quantity calculation section 404 calculates a feature quantity from the measurement data 411 and causes the storage section 41 to store the calculated feature quantity. Data which indicates the feature quantity and which the feature quantity calculation section 404 has caused the storage section 41 to store is feature quantity data 414. The feature quantity data 414 can include a feature quantity used for generation of training data 415. Hereinafter, a feature quantity used for generation of training data 415 is referred to as a training feature quantity.

The training feature quantity is a feature quantity used for machine learning of a stress level estimation model. Note, however, that generated training feature quantities are not all used for machine learning, and feature quantities selected by the first selection section 405 and the second selection section 406 from among the plurality of generated training feature quantities are used for generation of training data 415. Each of training feature quantities is associated with information indicating a modality of that feature quantity. For example, the information indicating a modality can indicate a type of measurement data (e.g., body motion data, heart rate data, perspiration data, or the like) from which the feature quantity has been obtained, or can indicate a classification such as physiological, behavioral, or psychological.

The feature quantity data 414 can also include a feature quantity used for estimation of a stress level. Hereinafter, the feature quantity used for estimation of a stress level is referred to as an estimation feature quantity. The estimation feature quantity is a feature quantity that has been generated from measurement data obtained, in a predetermined period (i.e., a time period for which a stress level is to be measured), from a subject whose stress level is to be estimated.

The first selection section 405 selects at least one training feature quantity corresponding to each of a plurality of modalities from among a plurality of training feature quantities based on respective utility evaluation results for the plurality of training feature quantities. Thus, a feature set is generated that includes at least one training feature quantity corresponding to each of the plurality of modalities. S22a through S22c in FIG. 3 are processes which are carried out by the first selection section 405. The first selection section 405 may evaluate utility for each of the training feature quantities by, for example, a filter method, or may evaluate utility for a combination of a plurality of training feature quantities by, for example, principal component analysis. Note that, in the case of using the filter method, the first selection section 405 may eliminate, when selecting a training feature quantity, a feature quantity having a high degree of similarity to another feature quantity, based on an index (such as a correlation coefficient or a mutual information content) that reflects a degree of similarity between feature quantities. This is because a training feature quantity having a high degree of similarity to another feature quantity is an obstacle to learning. In addition, for a similar purpose of eliminating similar feature quantities, the first selection section 405 may use a principal component analysis, an independent component analysis, or another technique having effects similar to these.

The second selection section 406 selects, based on a result of verifying estimation accuracy, a combination of training feature quantities for use in machine learning, the verification being carried out by applying combinations of training feature quantities included in the feature set generated by the first selection section 405 to the machine learning of the estimation model. S23 in FIG. 3 is a process carried out by the second selection section 406.

The training data generation section 407 generates training data by associating, as correct answer data, a stress level indicated in the stress level data 413 with a combination of training feature quantities which has been selected by the second selection section 406. Then, the training data generation section 407 causes the storage section 41 to store the generated training data as training data 415.

The training process section 408 generates, by training using the training data 415, an estimation model for which the training feature quantities selected by the second selection section 406 are used as explanatory variables and from which the stress level is obtained as an objective variable. S24 in FIG. 3 is a process that is carried out by the training process section 408. Then, the training process section 408 causes the storage section 41 to store the generated estimation model as an estimation model 416.

The estimation section 409 estimates the stress level of the subject using an estimation feature quantity that has been generated from measurement data of the subject. More specifically, the estimation section 409 calculates an estimation value of the stress level by inputting, into the estimation model 416, an estimation feature quantity included in the feature quantity data 414. S25 in FIG. 3 is a process that is carried out by the estimation section 409. Then the estimation section 409 causes the storage section 41 to store estimation result data 417 indicating a stress level estimation result.

(Flow of Estimation Model Generation Method)

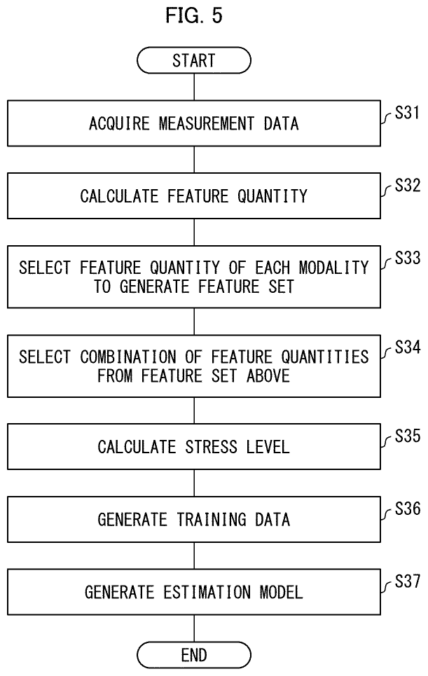

FIG. 5 is a flowchart illustrating a flow of an estimation model generation method according to the second example embodiment of the present invention. In the following descriptions, an example will be described in which an estimation model is generated using, as measurement data, triaxial acceleration data, heart rate data, and perspiration data of a subject which have been measured by the wearable terminal 7. The measurement data to be used can be measurement data of a single subject or can be pieces of measurement data of a plurality of subjects. However, it is preferable that the measurement data used is measurement data of a subject whose response to stress is close to that of a subject whose stress level is to be estimated. Further, in regard to each subject, it is assumed that a questionnaire has been answered for calculating a stress level during a period in which measurement data has been measured, and a result thereof is stored in the storage section 41 as questionnaire data 412. Further, all feature quantities in FIG. 5 are the above described training feature quantities. Therefore, in the description of FIG. 5, each of feature quantities is simply referred to as a feature quantity.

In S31, the measurement data acquisition section 401 acquires measurement data used for generation of an estimation model. As described above, measurement data acquired here is triaxial acceleration data, heart rate data, and perspiration data of the subject which have been measured by the wearable terminal 7. Then, the measurement data acquisition section 401 causes the storage section 41 to store the acquired measurement data as measurement data 411.

In S32, the feature quantity calculation section 404 calculates a feature quantity from the measurement data 411 that has been recorded in S31. Specifically, the feature quantity calculation section 404 calculates a plurality of types of feature quantities from each of the triaxial acceleration data, the heart rate data, and the perspiration data. The calculated feature quantities are stored in the storage section 41 as feature quantity data 414.

In S33, the first selection section 405 generates a feature set by selecting at least one feature quantity corresponding to each of the plurality of modalities from among a plurality of feature quantities based on respective utility evaluation results for the plurality of feature quantities which have been calculated in S32. For example, the first selection section 405 may evaluate utility of each of feature quantities generated from triaxial acceleration data by a filter method, and select a predetermined number of feature quantities whose evaluation result is higher. In this case, the first selection section 405 selects, for the feature quantities which have been generated from the heart rate data and for the feature quantities which have been generated from the perspiration data, a predetermined number of feature quantities whose evaluation result is higher, as with the case of the feature quantities which have been generated from the triaxial acceleration data. Thus, a feature set is generated which includes a predetermined number of feature quantities generated from each of the triaxial acceleration data, the heart rate data, and the perspiration data.

In S34, the second selection section 406 selects, based on a result of verifying estimation accuracy, a combination of feature quantities for use in the machine learning, the verification being carried out by applying combinations of feature quantities included in the feature set generated in S33 to the machine learning of the estimation model. For example, the second selection section 406 may select a combination of feature quantities by a wrapper method.

In S35, the stress level calculation section 403 calculates a stress level of the subject using the questionnaire data 412. Then, the stress level calculation section 403 causes the storage section 41 to store the calculated stress level as stress level data 413. Note that the process of S35 may be carried out before S31, or may be carried out concurrently with S31 through S34, as long as the process of S35 is carried out before S36.

In S36, the training data generation section 407 generates training data by associating, as correct answer data, a stress level which has been calculated in S35 and which is indicated in the stress level data 413 with the combination of feature quantities which has been selected in S34. Then, the training data generation section 407 causes the storage section 41 to store the generated training data as training data 415.

In S37, the training process section 408 generates a stress level estimation model by machine learning using the training data generated in S36. Note that S37 may include a series of processes in which a plurality of estimation models are generated, estimation accuracy of each of the generated estimation models is evaluated, and an ultimate estimation model is selected based on the evaluation results. Then, the training process section 408 causes the storage section 41 to store the generated estimation model as an estimation model 416. Thus, the estimation model generation method ends.

Note that, among the above described processes, S33 and S34 are the feature quantity selection method, S36 is the training data generation method, and S37 is the estimation model generation method. These processes can also be realized by a program. That is, a feature quantity selection program that causes a computer to carry out the processes of S33 and S34 is also encompassed in the scope of the present example embodiment. Similarly, a training data generation program that causes a computer to carry out the process (S36) of generating training data using the feature quantity selected in S34 is also encompassed in the scope of the present example embodiment. Moreover, an estimation model generation program that causes a computer to carry out the process (S37) of generating an estimation model using the training data generated in S36 is also encompassed in the scope of the present example embodiment.

(Stress Level Estimation Method)

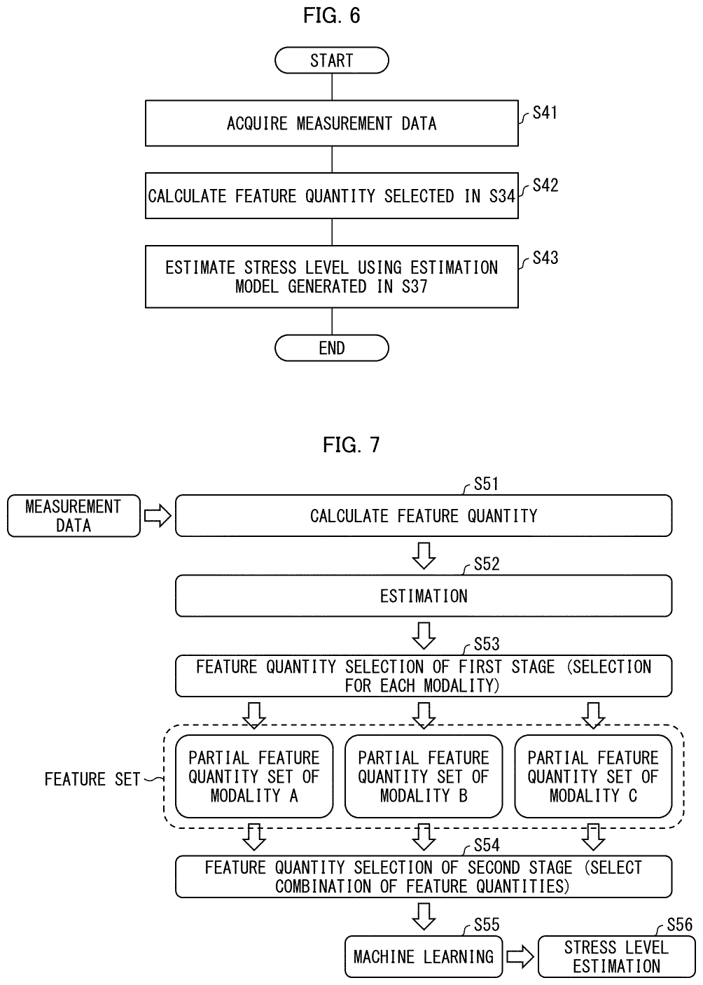

FIG. 6 is a flowchart illustrating a flow of a stress level estimation method according to the second example embodiment of the present invention. In the following descriptions, an example will be described in which a stress level of a subject in one month is estimated while using, as measurement data, triaxial acceleration data, heart rate data, and perspiration data which have been measured by the wearable terminal 7 for the one month. Note, however, that the measurement period can be less than one month or can be longer than one month. Further, the “feature quantity” in FIG. 6 is the above described estimation feature quantity. Therefore, in the description of FIG. 6, each of feature quantities is simply referred to as a feature quantity.

In S41, the measurement data acquisition section 401 acquires measurement data. As described above, measurement data acquired here is triaxial acceleration data, heart rate data, and perspiration data of the subject which have been measured by the wearable terminal 7 for one month. Then, the measurement data acquisition section 401 causes the storage section 41 to store the acquired measurement data as measurement data 411.

In S42, the feature quantity calculation section 404 calculates a feature quantity from the measurement data 411. The feature quantity calculated here is the feature quantity selected in S34 in FIG. 5, and is stored in the storage section 41 as feature quantity data 414.

In S43, the estimation section 409 estimates the stress level of the subject. Specifically, the estimation section 409 inputs, into the estimation model 416, the feature quantity which has been calculated in S42 and is indicated in the feature quantity data 414. This estimation model 416 is the estimation model generated in S37 in FIG. 5. Then, the estimation section 409 causes the storage section 41 to store an output value of the estimation model 416 as estimation result data 417. Note that the estimation section 409 may cause the output section 43 to output the estimated stress level. Thus, the stress level estimation method ends.

Note that the above processes can also be realized by a program. That is, a stress level estimation program that causes a computer to carry out the above described processes of S41 through S43 is also encompassed in the scope of the present example embodiment.

As described above, the information processing apparatus 4 according to the present example embodiment employs the configuration in which the first selection section 405 generates a feature set by carrying out, for each of modalities, evaluation of utility and selection of a feature quantity based on a result of the evaluation. According to the configuration, it is possible to generate a feature set that includes at least one feature quantity of each modality.

The information processing apparatus 4 according to the present example embodiment employs the configuration in which: the plurality of modalities include (i) a behavioral modality into which a feature quantity is classified which has been generated using measurement data that pertains to a behavior reflecting a stress state of a subject and (ii) a physiological modality into which a feature quantity is classified which has been generated using measurement data that pertains to a physiological phenomenon reflecting a stress state of the subject.

According to the configuration, it is easy to generate training data that includes both a feature quantity pertaining to a behavior of a subject and a feature quantity pertaining to a physiological phenomenon of the subject. By using such training data, it is possible to estimate a stress level while taking into consideration both the behavior and the physiological phenomenon of the subject. Therefore, according to the information processing apparatus 4 of the present example embodiment, it is possible to bring about an example advantage of estimating a stress level while taking into consideration both a behavior and a physiological phenomenon of a subject, in addition to the example advantage brought about by the information processing apparatus 1 according to the first example embodiment.

The training data generation method according to the present example embodiment includes: generating training data for use in the machine learning by associating, as correct answer data, a stress level of a subject with a combination of feature quantities which has been selected by the feature quantity selection method indicated in S33 and S34 in FIG. 5 (S36). Therefore, according to the training data generation method of the present example embodiment, it is possible to bring about an example advantage of generating training data that makes it possible to generate an estimation model having high robustness. Note that an execution subject of the training data generation method can be a processor included in the information processing apparatus 4 or can be a processor included in another apparatus. This also applies to the estimation model generation method and the stress level estimation method described below.

The estimation model generation method according to the present example embodiment includes: generating an estimation model by machine learning using training data which has been generated by the above training data generation method. Therefore, according to the estimation model generation method of the present example embodiment, it is possible to bring about an example advantage of generating an estimation model having high robustness.

The stress level estimation method according to the present example embodiment includes: estimating a stress level of a subject using an estimation model which has been generated by the above estimation model generation method. Therefore, according to the stress level estimation method of the present example embodiment, it is possible to bring about an example advantage of stably carrying out estimation with high accuracy.

### Third Example Embodiment

The following description will discuss a third example embodiment of the present invention in detail with reference to the drawings. FIG. 7 is a diagram illustrating overviews of a feature quantity selection method, a training data generation method, an estimation model generation method, and a stress level estimation method according to the present example embodiment. A difference from the second example embodiment is that, in the first stage of feature quantity selection, feature quantities are evaluated collectively without being classified according to modalities, and then a feature quantity of high evaluation is selected for each modality. The following description will discuss an example of causing the information processing apparatus 4 illustrated in FIG. 4 to carry out these methods.

In S51, as with S21 in FIG. 3, the feature quantity calculation section 404 calculates a feature quantity from measurement data pertaining to a stress level that indicates a degree of stress of a subject. The feature quantities calculated here include feature quantities of a plurality of modalities, as with the second example embodiment.

In S52, the first selection section 405 evaluates utility of each of the plurality of feature quantities which have been calculated in S51. Then, in S53, the first selection section 405 generates, based on the evaluation results in S52, a feature set by selecting at least one feature quantity corresponding to each of a plurality of modalities from among the plurality of feature quantities which have been calculated in S51.

For example, the first selection section 405 may select, for each of the plurality of modalities, a predetermined number of feature quantities whose evaluation result is higher. Note that the number of feature quantities to be selected for each modality may be fixed or may be changed in accordance with the evaluation result. For example, only a lower limit number of feature quantities to be selected for each modality may be determined in advance. In this case, the first selection section 405 only needs to select feature quantities whose evaluation result is higher regardless of the modality, after selecting feature quantities in the lower limit number for each modality. Thus, it is possible to select a feature quantity having higher utility while leaving a feature quantity of each modality.

As described above, by the processes in S52 and S53 (i.e., feature quantity selection method) in the present example embodiment also, it is possible to generate, as with the second example embodiment, a feature set including at least one feature quantity corresponding to each of a plurality of modalities. The processes of S54 through S56 are similar to the processes of S23 and S25 in FIG. 3, and therefore the descriptions thereof will not be repeated here. Note that S55 in FIG. 7 corresponds to the training data generation method and the estimation model generation method, and S56 corresponds to the stress level estimation method.

(Verification of Effect)

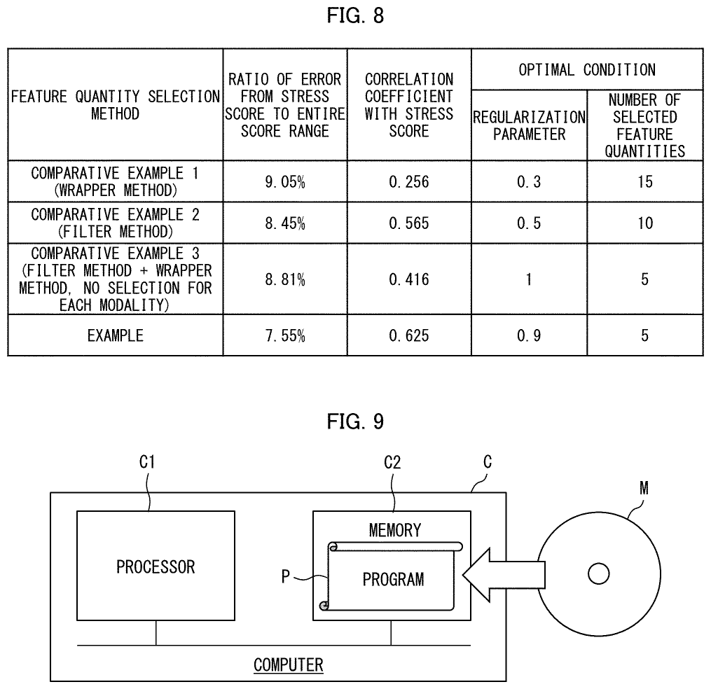

An experiment was conducted to verify the effect of the feature quantity selection method according to an example embodiment of the present invention. Results of the experiment are shown in FIG. 8. FIG. 8 is a diagram illustrating a result of an experiment for verifying an effect of the feature quantity selection method according to each of the example embodiments of the present invention.

In this experiment, leave-one-out cross-validation (LOOCV) was carried out on a total of 2292 feature quantities (training feature quantities) consisting of 936 pulse wave feature quantities generated from pulse wave data of a subject and 1356 acceleration feature quantities generated from triaxial acceleration data of the subject.

In each loop, an estimation model is generated by carrying out feature quantity selection from training data, and estimation accuracy of the generated estimation model is verified by test data. Verification of estimation accuracy was carried out based on an error (mean absolute error) and a correlation coefficient. It can be said that a lower error indicates higher estimation accuracy. Further, it can be said that a higher correlation coefficient indicates higher estimation accuracy.

As a stress score serving as correct answer data, a questionnaire result of 10 items of the perceived stress scale (PSS-10) was used. In this case, a score range is from 0 to 40. Therefore, for example, in a case where the error is 4, a ratio to an entire score range is 10%.

The feature quantity selection method was carried out in four patterns, that is, comparative example 1 (wrapper method), comparative example 2 (filter method), comparative example 3 (combination of filter method and wrapper method), and an example (combination of filter method and wrapper method). In the filter method in comparative example 3, 40 feature quantities were selected without considering the modality, the wrapper method was applied to the 40 feature quantities, and an optimal combination of feature quantities was selected. In contrast to this, in the filter method of the example, 20 feature quantities were selected for each of the modalities (pulse wave feature quantity and acceleration feature quantity) (40 feature quantities in total), the wrapper method was applied to the feature quantities, and an optimal combination of feature quantities was selected.

In order to find an optimum condition, the experiment was carried out while changing the regularization parameter from 0.1 to 1.0 in increments of 0.1 and changing the feature quantity selection number from 5 to 20 in increments of 5 in each of the feature quantity selection methods, and cases in each of which the best result was obtained in each feature quantity selection method were compared.

As illustrated in FIG. 8, the example in which a predetermined number of feature quantities were selected for each modality resulted in the highest accuracy in terms of both of the error and the correlation coefficient. This experimental result indicates that it is possible to estimate a stress level with high accuracy by using an estimation model which has been generated using feature quantities selected by the feature quantity selection method according to each of the example embodiments of the present invention.

### Software Implementation Example

The functions of part of or all of the information processing apparatuses 1 and 4 can be realized by hardware such as an integrated circuit (IC chip) or can be alternatively realized by software.

In the latter case, each of the information processing apparatuses 1 and 4 is realized by, for example, a computer that executes instructions of a program that is software realizing the foregoing functions. FIG. 9 illustrates an example of such a computer (hereinafter, referred to as “computer C”). The computer C includes at least one processor C1 and at least one memory C2. The memory C2 stores a program P for causing the computer C to function as the information processing apparatuses 1 and 4. In the computer C, the processor C1 reads the program P from the memory C2 and executes the program P, so that the functions of the information processing apparatuses 1 and 4 are realized.

As the processor C1, for example, it is possible to use a central processing unit (CPU), a graphic processing unit (GPU), a digital signal processor (DSP), a micro processing unit (MPU), a floating point number processing unit (FPU), a physics processing unit (PPU), a microcontroller, or a combination of these. The memory C2 can be, for example, a flash memory, a hard disk drive (HDD), a solid state drive (SSD), or a combination of these.

Note that the computer C can further include a random access memory (RAM) in which the program P is loaded when the program P is executed and in which various kinds of data are temporarily stored. The computer C can further include a communication interface for carrying out transmission and reception of data with other apparatuses. The computer C can further include an input-output interface for connecting input-output apparatuses such as a keyboard, a mouse, a display and a printer.

The program P can be stored in a non-transitory tangible storage medium M which is readable by the computer C. The storage medium M can be, for example, a tape, a disk, a card, a semiconductor memory, a programmable logic circuit, or the like. The computer C can obtain the program P via the storage medium M. The program P can be transmitted via a transmission medium. The transmission medium can be, for example, a communications network, a broadcast wave, or the like. The computer C can obtain the program P also via such a transmission medium.

[Additional Remark 1]

The present invention is not limited to the foregoing example embodiments, but may be altered in various ways by a skilled person within the scope of the claims. For example, the present invention also encompasses, in its technical scope, any example embodiment derived by appropriately combining technical means disclosed in the foregoing example embodiments.

[Additional Remark 2]

Some of or all of the foregoing example embodiments can also be described as below. Note, however, that the present invention is not limited to the following supplementary notes.

An information processing apparatus according to supplementary note 1 includes: a first selection means that generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a second selection means that selects, based on a result of verifying estimation accuracy, a combination of feature quantities for use in the machine learning, the verification being carried out by applying combinations of feature quantities included in the feature set to the machine learning of the estimation model. According to the configuration, it is possible to improve a feature quantity selection method for machine learning of a stress level estimation model.

The information processing apparatus according to supplementary note 2 employs, in addition to the configuration of supplementary note 1, the configuration in which: the first selection means generates the feature set by carrying out, for each of the plurality of modalities, evaluation of utility and selection of a feature quantity based on a result of the evaluation. According to the configuration, it is possible to generate a feature set that includes at least one feature quantity of each modality.

The information processing apparatus according to supplementary note 3 employs, in addition to the configuration of supplementary note 1 or 2, the configuration in which: the plurality of modalities include (i) a behavioral modality into which a feature quantity is classified which has been generated using measurement data that pertains to a behavior reflecting a stress state of a subject and (ii) a physiological modality into which a feature quantity is classified which has been generated using measurement data that pertains to a physiological phenomenon reflecting a stress state of the subject. According to the configuration, it is possible to estimate a stress level while taking into consideration both a behavior and a physiological phenomenon of a subject.

A feature quantity selection method according to supplementary note 4 includes: generating, by at least one processor, a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and selecting, by the at least one processor based on a result of verifying estimation accuracy, a combination of feature quantities for use in the machine learning, the verification being carried out by applying combinations of feature quantities included in the feature set to the machine learning of the estimation model. According to the configuration, it is possible to improve a feature quantity selection method for machine learning of a stress level estimation model.

A training data generation method according to supplementary note 5 includes: generating, by at least one processor, training data for use in machine learning by associating, as correct answer data, a stress level of a subject with a combination of feature quantities which has been selected by the feature selection method described in supplementary note 4. According to the configuration, it is possible to generate training data that makes it possible to generate an estimation model having high robustness.

An estimation model generation method according to supplementary note 6 includes: generating, by at least one processor, an estimation model by machine learning using training data which has been generated by the training data generation method described in supplementary note 5. According to the configuration, it is possible to generate an estimation model having high robustness.

A stress level estimation method according to supplementary note 7 includes: estimating, by at least one processor, a stress level of a subject using an estimation model which has been generated by the estimation model generation method described in supplementary note 6. According to the configuration, it is possible to stably carry out estimation with high accuracy.

A feature quantity selection program according to supplementary note 8 causes a computer to function as: a first selection means that generates a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a second selection means that selects, based on a result of verifying estimation accuracy, a combination of feature quantities for use in the machine learning, the verification being carried out by applying combinations of feature quantities included in the feature set to the machine learning of the estimation model. According to the configuration, it is possible to improve a feature quantity selection method for machine learning of a stress level estimation model.

[Additional Remark 3]

Furthermore, some of or all of the foregoing example embodiments can also be expressed as below. An information processing apparatus including at least one processor, the at least one processor carrying out: a process of generating a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and a process of selecting, based on a result of verifying estimation accuracy, a combination of feature quantities for use in the machine learning, the verification being carried out by applying combinations of feature quantities included in the feature set to the machine learning of the estimation model.

Note that the information processing apparatus can further include a memory. The memory can store a program for causing the at least one processor to carry out the process of generating a feature set and the process of selecting a combination of feature quantities for use in machine learning. The program can be stored in a computer-readable non-transitory tangible storage medium.

### REFERENCE SIGNS LIST

1: Information processing apparatus
11: First selection section
12: Second selection section
4: Information processing apparatus
405: First selection section
406: Second selection section

What is claimed is:

1. An information processing apparatus, comprising at least one processor, the at least one processor carrying out:
a first selection process of generating a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and
a second selection process of selecting, based on a result of verifying estimation accuracy, a combination of feature quantities for use in the machine learning, the verification being carried out by applying combinations of feature quantities included in the feature set to the machine learning of the estimation model.

2. The information processing apparatus according to claim 1, wherein:
in the first selection process, the at least one processor generates the feature set by carrying out, for each of the plurality of modalities, evaluation of utility and selection of a feature quantity based on a result of the evaluation.

3. The information processing apparatus according to claim 1, wherein:
the plurality of modalities include (i) a behavioral modality into which a feature quantity is classified which has been generated using measurement data that pertains to a behavior reflecting a stress state of a subject and (ii) a physiological modality into which a feature quantity is classified which has been generated using measurement data that pertains to a physiological phenomenon reflecting a stress state of the subject.

4. A feature quantity selection method, comprising:
generating, by at least one processor, a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and
selecting, by the at least one processor based on a result of verifying estimation accuracy, a combination of feature quantities for use in the machine learning, the verification being carried out by applying combinations of feature quantities included in the feature set to the machine learning of the estimation model.

5. A training data generation method, comprising:
generating, by at least one processor, training data for use in machine learning by associating, as correct answer data, a stress level of a subject with a combination of feature quantities which has been selected by a feature quantity selection method recited in claim 4.

6. An estimation model generation method, comprising:
generating, by at least one processor, an estimation model by machine learning using training data which has been generated by a training data generation method recited in claim 5.

7. A stress level estimation method, comprising:
estimating, by at least one processor, a stress level of a subject using an estimation model which has been generated by an estimation model generation method recited in claim 6.

8. A computer-readable non-transitory storage medium storing a program for causing a computer to carry out:
a first selection process of generating a feature set by selecting at least one feature quantity corresponding to each of a plurality of modalities from among a plurality of feature quantities based on respective utility evaluation results for the plurality of feature quantities which are usable for machine learning of an estimation model for estimating a stress level; and
a second selection process of selecting, based on a result of verifying estimation accuracy, a combination of feature quantities for use in the machine learning, the verification being carried out by applying combinations of feature quantities included in the feature set to the machine learning of the estimation model.
